(19) United States
(12) Patent Application Publication
(10) Pub. No.: US 2014/0142001 A1
(43) Pub. Date: May 22, 2014

(54) ORGANO-CASCADE CATALYSIS: ONE-POT PRODUCTION OF CHEMICAL LIBRARIES

(71) Applicant: THE TRUSTEES OF PRINCETON UNIVERSITY, PRINCETON, NJ (US)

(72) Inventor: David MACMILLAN, Princeton, NJ (US)

(73) Assignee: THE TRUSTEES OF PRINCETON UNIVERSITY, PRINCETON, NJ (US)

(21) Appl. No.: 14/165,079

(22) Filed: Jan. 27, 2014

Related U.S. Application Data
(63) Continuation of application No. 12/670,152, filed on Jan. 22, 2010, now abandoned.
(60) Provisional application No. 60/951,514, filed on Jul. 24, 2007.

(51) Int. Cl. C07C 45/61

(52) U.S. Cl. CPC C07C 45/61
USPC 506/27

(57) ABSTRACT

A method for production of a chemical library is provided, where the method involves: reacting, in a single vessel, a) a plurality, x, of aldehydes and/or ketones; and b) either (i) a plurality, y, of nucleophiles, (ii) a plurality, z, of electrophiles or both (i) and (ii); in the presence of c) a cascade catalyst capable of catalyzing reaction between said plurality of aldehydes and/or ketones and said plurality of nucleophiles, said plurality of electrophiles or both; to obtain a mixture of x-y β-nucleophile substituted aldehydes and/or ketones, x•z α-electrophile substituted aldehydes and/or ketones or x•y•z β-nucleophile substituted, α-electrophile substituted aldehydes and/or ketones; and the chemical libraries thus produced.

### BACKGROUND OF THE INVENTION

1. Field of Invention

The present invention relates to methods for the production of chemical libraries containing large numbers of different compounds in a single vessel, through the use of organo-cascade catalysis.

2. Discussion of the Background

The discovery of new synthetic technologies that allow rapid access to diverse molecular systems remains a preeminant goal for the chemical sciences. Within the realms of synthetic chemistry the traditional approach for the construction of complexity has focused on the ‘stop and go’ strategy, wherein chemical syntheses are designed on sequences of individual chemical transformations that are operated as stepwise processes. In contrast, Nature generates molecular complexity in continuous processes where enzymatic transformations are combined in highly regulated catalytic cascades that easily convert simple raw materials into complex molecular systems, a sophisticated chemical pathway that in laboratory language we term cascade catalysis.

Over the past years, the present inventors' laboratory has been involved in the development of the field of organocatalysis, a research area that relies on the use of small organic molecules to emulate the reactivity profiles of enzymes and metal-based catalysts. As part of these studies we introduced the imidazolidinone architecture (such as 1, Scheme 1) as a new class of secondary amine organocatalysts that are highly effective for enantioselective LUMO-lowering iminium activation (iminium 2). We have shown that the catalyst activated iminium ion 2 can enantioselectively intercept a diverse range of nucleophiles, and currently based on this strategy many different transformations have been developed for asymmetric synthesis. Recently, we discovered that the imidazolidinone catalysts are also highly effective for enantioselective HOMO-raising enamine activation (enamine 3) where the catalyst activated enamine 3 can enantioselectively intercept a wide variety of electrophiles. Based on this HOMO-raising enamine activation strategy many different transformations have been developed for asymmetric synthesis.

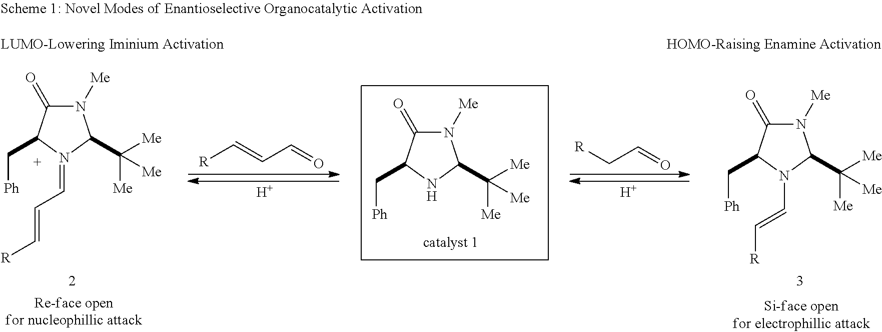

On this basis, the present inventors have shown that the conceptual blueprints of biosynthesis could be translated to a practical laboratory approach to cascade catalysis, wherein a single imidazolidinone catalyst could enable both iminium and enamine activation in a continuous catalytic process. This was accomplished by merging LUMO-lowering iminium activation and HOMO-raising enamine activation, using imidazolidinone catalyst 1, through which it has been shown that a large diversity of nucleophiles (furans, thiophenes, indoles, siloxyfurans, siloxyoxazoles, hydride sources) and electrophiles (fluorinating and chlorinating reagents) can efficiently undergo sequential addition with a wide array of α,β-unsaturated aldehydes to generate the cascade products in high chemical efficiency (Scheme 2), depending on the nucleophile and electrophile chosen.

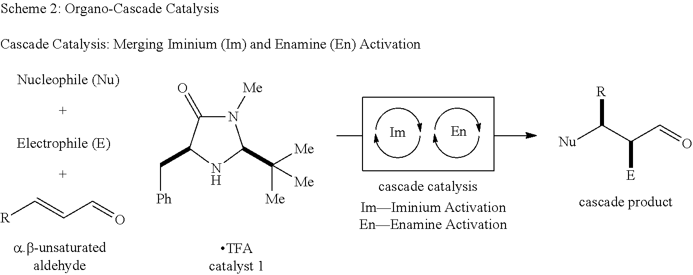

Chemical libraries are usually designed by chemists and chemo- or bio-informatics scientists and synthesized by organic or organometallic chemistry procedures. The method of chemical library generation usually depends on the project. Typically, chemical libraries comprise a large number of individual wells or vials, with a different chemical compound contained in each well or vial. The library is used to screen against a particular substrate or ligand in order to determine activity of the individual compounds, in order to select a compound with affinity for the desired activity. The generation of such libraries can be a painstaking process, requiring the generation of each individual compound separately, either directly in each of the vials or wells, or if outside the wells or vials, requiring the step of placing each compound into its well or vial.

More recently, methods for screening mixtures of compounds for biological activity have been developed, including, but not limited to, the CHEMETICS technology of Nuevolution, in which the target binding agent can be immobilized on a column, and a mixture of compounds, each of which have a unique DNA tag, applied to the column to determine which bind with the target binding agent (as disclosed in various of US Published Applications 2003/0143561; 2004/0049008; 2005/0247001; 2006/0099589; 2006/0099592; 2006/0234231; 2006/0246450; 2006/0269920; and 2007/0026397); or the assays described in U.S. Pat. Nos. 5,306,619 and 7,041,509, the contents of which are hereby incorporated by reference. Many such assays are affinity type assays, using ligand-receptor type binding to determine affinity of compounds in the mixture for a particular biological target. The binding agents are frequently proteins, nucleic acids and other biological products. Since such biological screening reactions are often confounded by the presence of metal based byproducts and impurities, the use of such screening often requires extensive purification procedures be performed on the chemical library components prior to use.

Accordingly, a method is needed that can generate a chemical library containing large numbers of compounds, preferably in a single vessel, and which is free of metals and reagent byproducts that can poison the substrate/ligand used to screen the chemical library and thus does not require further purification processes.

### SUMMARY OF THE INVENTION

Accordingly, one object of the present invention is to provide a method for the production of chemical libraries using cascade catalysis.

A further object of the present invention is to provide a method for the efficient production of chemical libraries containing large numbers of compounds in the same vessel.

A further object of the present invention is to provide large chemical libraries produced by the method.

These and further objects of the present invention, either individually or in combinations thereof, have been satisfied by the discovery of a method for production of a chemical library, comprising:
reacting, in a single vessel,
a) a plurality, x, of aldehydes and/or ketones; and
b) either (i) a plurality, y, of nucleophiles, (ii) a plurality, z, of electrophiles or both (i) and (ii); in the presence of
c) a cascade catalyst capable of catalyzing reaction between said plurality of aldehydes and/or ketones and said plurality of nucleophiles, said plurality of electrophiles or both;
to obtain a mixture of x•y β-nucleophile substituted aldehydes and/or ketones, x•z α-electrophile substituted aldehydes and/or ketones or x•y•z β-nucleophile substituted, α-electrophile substituted aldehydes and/or ketones; and the chemical libraries produced thereby.

### DETAILED DESCRIPTION OF THE INVENTION

The present invention relates to a method for the preparation of chemical libraries for biological screening that is based on organo-cascade catalysis, in order to avoid the introduction of metallic based reagents and reagent byproducts that have an adverse impact on the biological screen to be performed. The present invention is highly amenable to large library synthesis and provides several advantages over the current approaches used for the generation of molecular complexity. Firstly, the organo-cascade sequence, preferably involving iminium-enamine catalytic cycles rapidly transforms commercially available α,β-unsaturated aldehydes and ketones into complex products in a single chemical operation. Further, the cascade products can be used as intermediates in other chemical transformations to introduce chemical diversity and extend the range of chemical functionalities present in the final products. Another major benefit of the present invention organo-cascade catalysis strategy is that the cascade sequence uses a simple organic molecule to catalyze the process as opposed to using metal-based catalysts. This represents an important advantage since the resulting chemical libraries will not be plagued by metal byproducts that will compel the introduction of a tedious purification operation before biological testing.

Unless otherwise indicated, the invention is not limited to specific molecular structures, substituents, synthetic methods, reaction conditions, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a catalyst” includes a single catalyst as well as a combination or mixture of two or more catalysts, reference to “a substituent” encompasses a single substituent as well as two or more substituents, and the like.

In this specification and in the claims that follow, reference will be made to a number of terms, which shall be defined to have the following meanings:

The term “alkyl” as used herein refers to a linear, branched, or cyclic saturated hydrocarbon group typically although not necessarily containing 1 to about 24 carbon atoms, preferably 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, and the specific term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The terms “cyclic” and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

The terms “halo” and “halogen” are used in the conventional sense to refer to a chloro, bromo, fluoro or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated and unsaturated species, such as alkyl groups, alkenyl groups, aryl groups, and the like. The term “lower hydrocarbyl” intends a hydrocarbyl group of 1 to 6 carbon atoms, preferably 1 to 4 carbon atoms, and the term “hydrocarbylene” intends a divalent hydrocarbyl moiety containing 1 to about 30 carbon atoms, preferably 1 to about 24 carbon atoms, most preferably 1 to about 12 carbon atoms, including linear, branched, cyclic, saturated and unsaturated species. The term “lower hydrocarbylene” intends a hydrocarbylene group of 1 to 6 carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and “heterohydrocarbylene” refer to hydrocarbylene in which at least one carbon atom is replaced with a heteroatom. Unless otherwise indicated, the term “hydrocarbyl” and “hydrocarbylene” are to be interpreted as including substituted and/or heteroatom-containing hydrocarbyl and hydrocarbylene moieties, respectively.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including C₂-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O—CO-alkyl) and C₆-C₂₄ arylcarbonyloxy (—O—CO-aryl)), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₅-C₂₄ aryl)-substituted carbamoyl (—(CO)—NH-aryl), di-(C₅-C₂₄ aryl)-substituted carbamoyl (—(CO)—N(C₅-C₂₄ aryl)₂), di-N—(C₁-C₂₄ alkyl), N—(C₅-C₂₄ aryl)-substituted carbamoyl, thiocarbamoyl (—(CS)—NH₂), mono-(C₁-C₂₄ alkyl)-substituted thiocarbamoyl (—(CO)—NH(C₁-C₂₄ alkyl)), di-(C₁-C₂₄ alkyl)-substituted thiocarbamoyl (—(CO)—N(C₁-C₂₄ alkyl)₂), mono-(C₅-C₂₄ aryl)-substituted thiocarbamoyl (—(CO)—NH-aryl), di-(C₅-C₂₄ aryl)-substituted thiocarbamoyl (—(CO)—N(C₅-C₂₄ aryl)₂), di-N—(C₁-C₂₄ alkyl), N—(C₅-C₂₄ aryl)-substituted thiocarbamoyl, carbamido (—NH—(CO)—NH₂), cyano(—CN), cyanato (—O—CN), thiocyanato (—S—CN), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C6-C24 arylamido (—NH—(CO)-aryl), imino (—CR═NH where R=hydrogen, C₁-C₂₄ alkyl, C—C₂₄ aryl, C₆-C₂₄ alkaryl, C₆-C₂₄ aralkyl, etc.), C₂-C₂₀ alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₀ alkyl, C₅-C₂₄ aryl, C6-C₂₄ alkaryl, C₆-C₂₄ aralkyl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

In the molecular structures herein, the use of bold and dashed lines to denote particular conformation of groups follows the IUPAC convention. A bond indicated by a broken line indicates that the group in question is below the general plane of the molecule as drawn (the “α” configuration), and a bond indicated by a bold line indicates that the group at the position in question is above the general plane of the molecule as drawn (the “β” configuration).

The process of the present invention can be represented by the following equations (i) and (ii):

x(α,β-unsaturated carbonyl compounds)+y(nucleophiles)+catalyst→x•y(β-nucleophile substituted carbonyl compounds) (i)

x•y(β-nucleophile substituted carbonyl compounds)+z(electrophiles)+catalyst→x•y•z[(β-nucleophile substituted)−(α-electrophile substituted) carbonyl compounds] (ii)

Or the following equation (iii):

x(carbonyl compounds)+z(electrophiles)+catalyst→x•z(α-electrophile substituted carbonyl compounds) (iii)

where x, y and z represent the number of different carbonyl compounds, nucleophiles and electrophiles, respectively. The resulting product mixture will contain a distribution of x•y, x•z or x•y•z permutations of compounds, depending on whether equation (i), (ii) or (iii) is being performed, and in amounts that differ only as a result of the reactivities of the individual starting materials reactivities in the catalyzed reaction. In general, each of x, y and z represents, independently, a plurality of each type of reactant, preferably an integer in the range from 5 to 5000, more preferably an integer in the range from 10 to 1000, most preferably an integer in the range from 10 to 100.

The starting material aldehydes and/or ketones of the embodiments of the present invention, using both enamine and iminium activations or iminium activation alone, can be any α,β-unsaturated aldehyde or ketone, preferably having one of the structures below:

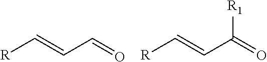

where R and R₁ are each, independently, selected from alkyl, alkenyl, alkynyl, cycloalkyl, aryl, heteroaryl, alkoxy, aryloxy, heteroaryloxy, alkaryl, or aralkyl, each of which may be unsubstituted or substituted by one or more substituents.

For the embodiment of the present invention using only the enamine activation to produce α-substituted aldehydes and/or ketones, the starting material aldehydes and/or ketones can be any aldehyde or ketone, preferably one having one of the structures below:

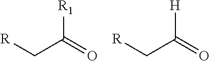

where R and R₁ are as defined above.

The catalysts useful in the present invention cascade catalysis reactions include any of the organic catalysts described in U.S. Pat. Nos. 6,307,057; 6,369,243; 6,515,137; 6,534,434; 6,784,323; and 7,173,139; or US Published applications 2003/0220507 or 2006/0189830 (collectively, “the MacMillan patents”). The entire contents of each of these issued US patents and published US applications is hereby incorporated by reference.

The nucleophiles that can be used in the present invention can be any source of a nucleophilic group, preferably a source that is free of metallic elements. Suitable nucleophiles include, but are not limited to, those described in the MacMillan patents above, as well as indoles, pyrroles, furans, thiophenes, anilines, 2-siloxy-furans 2-siloxy-oxazoles, silyl-enol ethers, amines, alcohols, thiols, cyclic dienes, acyclic dienes and Hantzch esters, which may be substituted or unsubstituted.

The electrophiles that can be used in the present invention can be any source of an electrophilic group, preferably a source that is also free of metallic elements. Suitable electrophiles include, but are not limited to, those described in the MacMillan patents above, as well as 2,3,4,5,6,6-hexachlorocyclohexa-2,4-dienone (source of Cl), phenyl fluoro(phenylsulfonyl) carbamate (source of F), 1-bromopyrrolidine-2,5-dione (source of Br), 1-iodopyrrolidine-2,5-dione (source of I), aldehydes and ketones (aldol reactions), imines (Mannich reactions), enals and enones (Michael reactions), nitrosobenzene (source of O), and azodicarboxylates (source of N).

When performing the cascade catalysis reaction of the present invention, the starting carbonyl compounds, nucleophiles and electrophiles are combined in a reaction vessel along with the cascade catalyst. The order of addition is preferably starting carbonyl compounds, followed by nucleophiles (if used), then electrophiles (if used). The ratio of total carbonyl compounds:total nucleophiles:total electrophiles can be altered over a wide range, but is preferably close to 1:1:1 in order to obtain the most evenly diversified mixture of products. The cascade catalyst is used in an amount of from 5 to 20 mol percent, based on total carbonyl compounds present, preferably in an amount from 5 to 15 mol percent, most preferably from 7 to 12 mol percent.

The mixture is allowed to react for a period of from several hours to several days, preferably from 2-3 days, at temperatures from −60° C. to room temperature (approx. 25-30° C.), preferably from −40° C. to room temperature. One distinct advantage of the present invention chemical libraries is that following the reaction, no additional workup procedures are required, and no separation or purification of compounds is necessary (unless, of course, it is desired to further functionalize the resulting compounds as noted below). Typically the only post-reaction step may be passing the reaction mixture through a chromatography column filled with a sorbent such as silica, using a polar solvent such as diethyl ether or tetrahydrofuran, followed by concentrating the eluent.

Considering, for example, an embodiment of the present invention where the number of individual starting materials is set at 1000 α,β-unsaturated aldehydes, 1000 nucleophiles, and 10 electrophiles, after the cascade sequence (first chemical operation) the reaction vessel could hold a minimum of 10,000,000 individual compounds (Scheme 3).

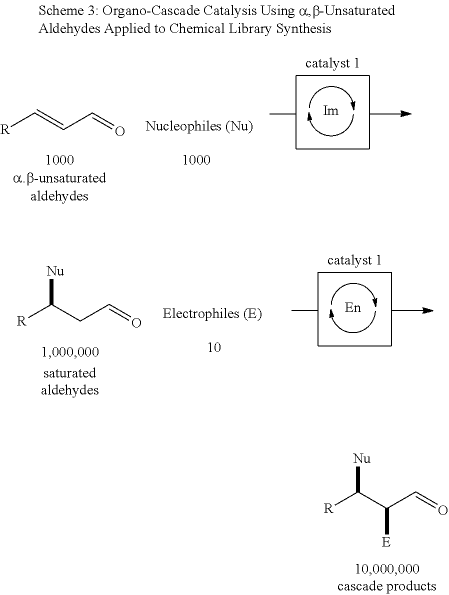

These cascade products are unique in that the aldehyde or ketone carbonyl functionality can be used as a synthetic handle to further introduce molecular complexity. A wide range of practical synthetic transformations can be performed on such mixtures, so long as the transformation reaction does not adversely affect or react with other portions of the compounds. Preferably, these additional transformations include, but are not limited to, reactions of reductive amination, esterification, and amidation. Each of these transformations can be used to convert the cascade products into chemical functionalities prevalent in pharmaceutical agents (amines, esters, and amides), concomitantly introducing another point of diversity (Scheme 4).

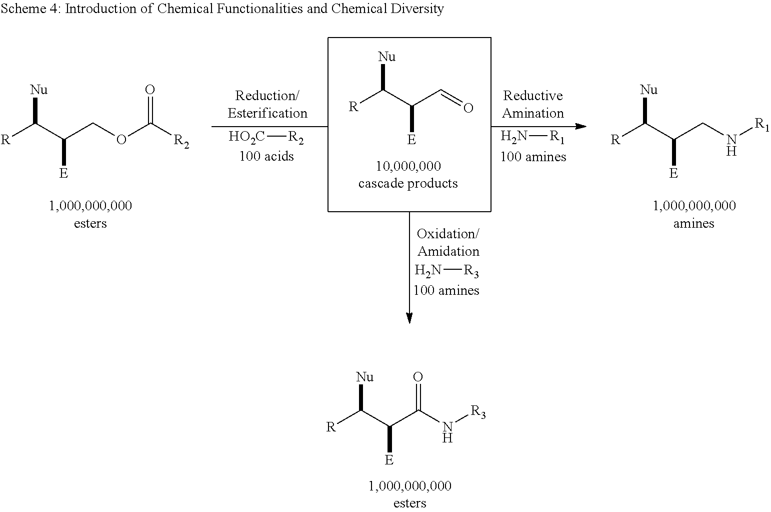

By use of a plurality of reagents for each of these types of transformations, it is possible to introduce even further diversity into the chemical library. For example, use of 100 reagents per transformation (100 amines for reductive amination, 100 carboxylic acids for esterifications, or 100 amines for amidations) would further extend the library to a minimum of 1,000,000,000 compounds. By splitting the original chemical library produced after cascade catalysis into three portions and performing each of the above further transformations, one obtains three chemical libraries having a total of 3,000,000,000 compounds which can be rapidly screened using the mixture screening technology of Merck. It is important to note that these large chemical libraries are prepared in just two chemical operations.

As noted above for the use of α,β-unsaturated aldehydes, a further embodiment of the present invention uses α,β-unsaturated ketones as starting materials in the iminium-enamine cascade sequence (Scheme 5). Therefore, similar chemical libraries of cascade products based on the ketone architecture can be generated, and further points of diversity can be introduced by identifying a variety of transformations for the cascade ketone products.

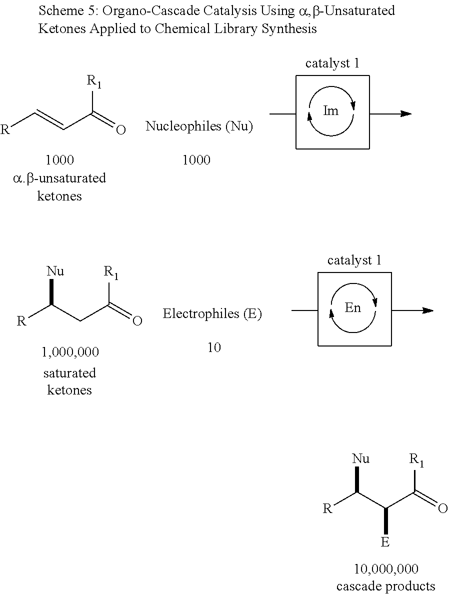

Ideally, any further transformations would use metal free reactants, in order to avoid introduction of metals and metal compounds into the library. However, certain transformations can be performed using metal based reactants, so long as the workup after the reaction is relatively simple for removing the metallic compounds. In this embodiment of the present invention, the process preferably further comprises at least one step selected from the group consisting of:
reductive amination of said mixture of x•y β-nucleophile substituted aldehydes and/or ketones, x•z α-electrophile substituted aldehydes and/or ketones or x•y•z β-nucleophile substituted, α-electrophile substituted aldehydes and/or ketones using a plurality, p, of amines;
oxidative amidation of said mixture of x•y β-nucleophile substituted aldehydes and/or ketones, x•z α-electrophile substituted aldehydes and/or ketones or x•y•z β-nucleophile substituted, α-electrophile substituted aldehydes and/or ketones using a plurality, q, of amines;
reduction and esterification of said mixture of x•y β-nucleophile substituted aldehydes and/or ketones, x•z α-electrophile substituted aldehydes and/or ketones or x•y•z β-nucleophile substituted, α-electrophile substituted aldehydes and/or ketones using a reducing agent and a plurality, r, of carboxylic acids;
Grignard addition to said mixture of x•y β-nucleophile substituted aldehydes and/or ketones, x•z α-electrophile substituted aldehydes and/or ketones or x•y•z β-nucleophile substituted, α-electrophile substituted aldehydes and/or ketones using a plurality, s, of hydrocarbyl Grignard reagents; and
   Wittig reaction of said mixture of x•y β-nucleophile substituted aldehydes and/or ketones, x•z α-electrophile substituted aldehydes and/or ketones or x•y•z β-nucleophile substituted, α-electrophile substituted aldehydes and/or ketones using a plurality, t, of hydrocarbyl phosphorous-based Wittig reagents.

Other transformations could be performed besides those specified above, including, but are not limited to, imidization (forming C═N bonds from the carbonyl), etc. It is preferred for these additional transformations that the reaction being performed does not affect the functionality or stereochemistry elsewhere within the compounds.

In the present invention, the cascade catalysis production of chemical libraries is preferably performed using either enamine activation combined with reaction with electrophiles (using an aldehyde or ketone having no α,β-unsaturation as in Scheme 6), iminium activation of α,β-unsaturated aldehydes or ketones combined with reaction with nucleophiles, or both the enamine and iminium activation of α,β-unsaturated aldehydes or ketones combined with reaction with both electrophiles and nucleophiles. This presents the opportunity to prepare chemical libraries of α-substituted aldehydes and ketones (see Scheme 6 below), β-substituted aldehydes and ketones (such as the first half of Scheme 5 above), or the (β-nucleophile substituted)-(α-electrophile substituted) aldehydes and ketones of the full cascade catalysis sequence shown above in Schemes 3 and 5.

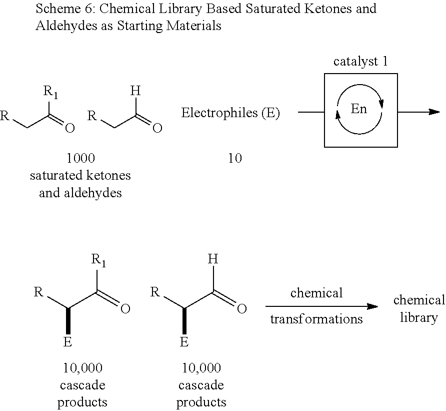

Having generally described this invention, a further understanding can be obtained by reference to certain specific examples which are provided herein for purposes of illustration only and are not intended to be limiting unless otherwise specified.

### Examples

### General Procedure for Organo-Cascade Catalysis Sequence

To a 200 mL reaction vessel equipped with a stirring bar and charged with catalyst (20 mol %) is added dichloromethane and cooled to −50° C. To this solution is added a preformed mixture of the α,β-unsaturated aldehydes in dichloromethane, followed by the nucleophiles (1 equivalent per nucleophile). The reaction mixture is allowed to stir at −50° C. for 16 hours, then at −40° C. for 12 hours. At this point the electrophiles (1 equivalent per electrophile) are added and the reaction mixture is stirred at −20° C. for 12 hours and 0° C. for 1 hour. The crude reaction mixture is then passed through a short plug of silica gel with diethyl ether, and concentrated under reduced pressure. The concentrated reaction mixture is then divided into three equal parts and each part is subjected to a different reaction as outlined below.

### General Procedure for Reduction/Esterification

The crude reaction mixture is taken up in ethanol, cooled to 0° C. and stirred for 10 minutes. To this solution is added sodium borohydride (NaBH₄) (3 equivalents per aldehyde) and the reaction mixture is allowed to stir for 1 hour at 0° C. The reaction mixture is then quenched by addition of cold water, washed with brine and extracted with dichloromethane. The organic layer is dried with MgSO₄, filtered and concentrated under reduced pressure. The crude reaction mixture is then added to a pre-cooled solution (0° C.) of five carboxylic acids (1 equivalent) dicyclohexylcarbodiimide (DCC) (1.5 equivalents per carboxylic acid) in dicholomethane. The reaction mixture is allowed to stir for 4 hours while warming to room temperature. The urea by-products are filtered and washed with dichloromethane, and filtered solution is concentrated under reduced pressure and submitted for LC/MS (Liquid Chromatography/Mass Spectrometry) analysis.

### General Procedure for Reductive Amination

The crude reaction mixture is taken up in ethanol, and three aniline (1.5 equivalents) derivatives are added. The reaction mixture is allowed to stir for 1 hour then sodium borohydride (NaBH₄) (2 equivalents per imine) is added. The reaction mixture is allowed to stir for 30 mins and quenched with water, washed with brine and extracted with dichloromethane. The organic layer is dried with MgSO₄, filtere, concentrated under reduced pressure and submitted for LC/MS (Liquid Chromatography/Mass Spectrometry) analysis.

### General Procedure for Grignard Additions

To a pre-cooled (10° C.) mixture of five alkyl and aryl Grignard reagents (1.2 equivalents) in diethylether is added a solution of the crude reaction mixture (dissolved in diethylether) over 30 mins. The reaction mixture is allowed to stir at 10° C. for 1 hour then poured into crushed ice, washed with brine, and extracted with diethylether. The organic layer is dried with MgSO₄, filtered, concentrated under reduced pressure and submitted for LC/MS (Liquid Chromatography/Mass Spectrometry) analysis.

Obviously, additional modifications and variations of the present invention are possible in light of the above teachings. It is therefore to be understood that within the scope of the appended claims, the invention may be practiced otherwise than as specifically described herein.

1. A method for production of a chemical library, comprising:
reacting, in a single vessel,
a) a plurality, x, of aldehydes and/or ketones; and
b) either (i) a plurality, y, of nucleophiles, (ii) a plurality, z, of electrophiles or both (i) and (ii); in the presence of
c) a cascade catalyst capable of catalyzing reaction between said plurality of aldehydes and/or ketones and said plurality of nucleophiles, said plurality of electrophiles or both;
to obtain a mixture of x•y β-nucleophile substituted aldehydes and/or ketones, x•z α-electrophile substituted aldehydes and/or ketones or x•y•z β-nucleophile substituted, α-electrophile substituted aldehydes and/or ketones.
